(19) United States
(12) Patent Application Publication
(10) Pub. No.: US 2025/0049511 A1
(43) Pub. Date: Feb. 13, 2025

(54) MAGNETIC RESONANCE TOMOGRAPH

(71) Applicant: Siemens Healthineers AG, Forchheim (DE)

(72) Inventor: Arne Hengerer, Möhrendorf (DE)

(21) Appl. No.: 18/797,490

(22) Filed: Aug. 7, 2024

(30) Foreign Application Priority Data
Aug. 8, 2023 (EP) 23190298.2

(51) Int. Cl. A61B 34/20; A61B 5/00; A61B 5/055; A61B 34/10

(52) U.S. Cl. CPC A61B 34/20; A61B 5/055; A61B 5/704; A61B 34/10; A61B 2034/107; A61B 2034/2051

(57) ABSTRACT

A magnetic resonance tomograph includes a patient table, a patient receiving region, a feed unit that is configured to guide a medical instrument, and a rail system for guiding the feed unit. The rail system runs extensively on an interior side along an inner side of the patient receiving region). The patient table is configured to position a patient in the patient receiving region. The patient table is configured to be movable such that the medical instrument may be guided along a trajectory in the patient with assistance of a movement of the patient table.

This application claims the benefit of European Patent Application No. EP 23190298.2, filed on Aug. 8, 2023, which is hereby incorporated by reference in its entirety.

### BACKGROUND

The present embodiments relate to a magnetic resonance tomograph, a method for planning a movement of a medical instrument in a patient receiving region of a magnetic resonance tomograph, a planning system configured to perform the method, a computer program product, and a computer-readable storage medium.

It is known to monitor an intervention on a patient using medical imaging performed with a magnetic resonance tomograph. This is also known as an image-assisted intervention. To this end, the patient is positioned in a patient receiving region in the magnetic resonance tomograph. The patient receiving region may be formed by an enclosure of the magnetic resonance tomograph and may be cylindrical. The intervention may be carried out with a medical instrument that is inserted into the patient. Such an intervention may, for example, be image-monitored biopsy sampling via a biopsy needle and/or ablation of a lesion using a catheter. The biopsy needle or catheter is to be inserted into the patient while the patient is positioned in the patient receiving region of the magnetic resonance tomograph for medical imaging. Access to the patient within the patient receiving region is impeded.

It is known to insert the medical instrument into the patient manually. In other words, it is known for the medical instrument to be inserted into the patient by medical personnel. Due to the impeded access, the member of medical personnel is to meet specific physical prerequisites (e.g., a minimum size). Further, the ergonomics for the medical personnel on insertion of the medical instrument are poor.

Alternatively, intervention robots that move relative to the patient in the patient receiving region and in so doing insert the medical instrument into the patient are known. However, this entails there being sufficient space between the patient and a patient-facing internal wall of the patient receiving region. Since the diameter of the patient receiving region is typically rather small, this condition is not met for every patient. An intervention robot thus cannot be used for all patients.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a magnetic resonance tomograph that enables ease of handling of a medical instrument in an image-assisted intervention within a patient receiving region is provided.

A magnetic resonance tomograph, a method for planning a movement of a medical instrument in a patient receiving region of a magnetic resonance tomograph, a planning system configured to perform the method, a computer program product, and a computer-readable storage medium are provided.

Corresponding functional features of the method are, for example, formed by corresponding substantive modules.

Independent of the grammatical term usage, individuals with male, female, or other gender identities are included within the term.

The present embodiments relate to a magnetic resonance tomograph including a patient table, a patient receiving region, a feed unit, and a rail system. The feed unit is configured to guide a medical instrument. The rail system runs extensively on an interior side of an inner side of the patient receiving region. The patient table is configured to position a patient in the patient receiving region. The patient table is configured to be movable such that the medical instrument may be guided along a trajectory in the patient with the assistance of a movement of the patient table.

The magnetic resonance tomograph may include a medical and/or diagnostic magnetic resonance tomograph that is designed or configured to acquire medical and/or diagnostic image data (e.g., medical and/or diagnostic magnetic resonance image data) from a patient. To this end, the magnetic resonance tomograph includes a magnet unit. The magnet unit of the magnetic resonance tomograph may include a detector unit for acquiring the medical and/or diagnostic image data. The magnet unit may include a main magnet, a gradient coil unit, and a radio-frequency antenna unit. The radio-frequency antenna unit is fixedly arranged within the magnet unit and designed and/or configured to emit an excitation pulse. The magnetic resonance tomograph has local radio-frequency coils that are arranged around the region to be examined of the patient in order to acquire the magnetic resonance signals.

The main magnet of the magnet unit is configured to generate a homogeneous main magnetic field with a defined or specific magnetic field strength, such as, for example, with a defined or specific magnetic field strength of 3 T or 1.5 T, etc. The main magnet is, for example, configured to generate a strong, constant, and homogeneous main magnetic field. The homogeneous main magnetic field may be arranged and/or found within the patient receiving region of the magnetic resonance tomograph. The gradient coil unit is configured to generate magnetic field gradients that are used for spatial encoding during imaging.

The patient receiving region is designed or configured to accommodate the patient (e.g., the region to be examined of the patient) for a medical magnetic resonance examination. To this end, the patient receiving region is, for example, of cylindrical construction or cylindrically surrounded by the magnet unit. The patient receiving region may have a circular or elliptical cross-section. To this end, the magnet unit has a housing unit enclosure that at least partially surrounds the patient receiving region. The enclosure surrounding the patient receiving region may also be formed integrally or in one piece with the side of the radio-frequency antenna unit facing the patient receiving region of the magnet unit or also be formed separately from the radio-frequency antenna unit of the magnet unit.

A field of view (FOV) and/or isocenter of the magnetic resonance tomograph may be arranged within the patient receiving region. The FOV may include an acquisition region of the magnetic resonance tomograph, within which conditions for acquiring medical image data (e.g., magnetic resonance image data), such as, for example, a homogeneous main magnetic field prevail. The isocenter of the magnetic resonance tomograph may include the region and/or point within the magnetic resonance tomograph that has the optimum or ideal conditions for acquiring medical image data (e.g., magnetic resonance image data). The isocenter, for example, includes the most homogeneous region of the magnetic field within the magnetic resonance tomograph.

The magnetic resonance tomograph includes a patient positioning apparatus. The patient positioning apparatus is configured for positioning and/or placing the patient for a magnetic resonance examination. The patient positioning apparatus includes the patient table that is configured to be advanceable into the patient receiving region. For a magnetic resonance examination, the patient is positioned on the patient table such that, once the patient table has been arranged within the patient receiving region, the region to be examined is arranged and/or positioned within the isocenter of the patient receiving region.

The magnetic resonance tomograph has a communication unit for communication and/or exchange of information between the patient and the medical operator during a magnetic resonance examination. On the user side, the communication unit may have a communication element, such as, for example, a communication console for inputting and/or outputting communication data, such as, for example, information. On the patient side, the communication unit further likewise has at least one communication element. For example, the communication unit has a visual or auditory communication element.

The rail system is arranged on an inner side of the patient receiving region. In other words, the rail system is arranged on a side of the enclosure that faces the patient during an examination. The rail system is arranged extensively on the interior side. In other words, the rail system is arranged tangentially to a surface of the enclosure. In other words, the rail system extends at least in part along a circumference of the inner side of the patient receiving region.

The feed unit is configured to guide or hold the medical instrument. The medical instrument may be fastened to the feed unit. The feed unit may be moved, displaced, or guided along the rail system. For example, the feed unit may be moved along the rail system with the assistance of rollers and/or bearings. The rail system, for example, specifies guidance (e.g., a direction), in which the feed unit is movable.

The feed unit may optionally be configured to tilt and/or rotate the medical instrument. For example, the medical instrument may be configured to be rotatable about an axis perpendicular to the rail system. Alternatively or additionally, the feed unit may be configured to tilt the medical instrument relative to the axis.

The medical instrument may, for example, be configured to carry out a medical intervention. For example, the medical instrument may be configured to be inserted into a patient. For example, the medical instrument may be configured to be moved or guided along a trajectory in a patient positioned on the patient table.

The trajectory may, for example, be straight and/or curved. For example, the trajectory may be the shortest path from a puncture site or entry point of the medical instrument into the patient to a location of an intervention or a target position in the patient. Alternatively, the trajectory may be curved. Alternatively, the trajectory may extend along or within a blood vessel of the patient.

The intervention may, for example, be a biopsy and/or an ablation and/or a catheter intervention. The medical instrument may, for example, include a biopsy needle, a catheter, or a high-intensity, focused ultrasound probe.

The patient table is configured such that the medical instrument may be moved along the trajectory by movement of the patient table. In other words, by moving the patient table, the patient placed on the patient table may be moved relative to the feed unit and thus relative to the medical instrument fastened to the feed unit such that the medical instrument may be moved along the trajectory in the patient.

Such a magnetic resonance tomograph provides that there is no need for manual movement of the medical instrument by medical personnel. In this manner, the physical prerequisites placed on the medical personnel may be made less stringent. Non-ergonomic situations for the medical personnel are avoided. By the patient being moved relative to the medical instrument, it is possible to overcome the problem of insufficient space in the patient receiving region. For example, it is thus no longer necessary to provide very much space for the medical instrument in the patient receiving region since it is not necessary to move the medical instrument itself toward the patient and, for example, in the patient.

According to one aspect of the present embodiments, the patient table includes at least three axes. The axes are configured such that the medical instrument is movable along the trajectory by movement of the patient table. The movement of the patient table is produced by a linear combination of a movement along the axes and/or a rotation about at least one of the axes.

The patient table is, for example, movable or displaceable along the three axes. For example, the patient table is movable or displaceable along the three axes in the patient receiving region.

The patient table may be configured to be rotatable about at least one of the three axes.

The axes are oriented such that, by moving the patient table along or rotating the patient table about the axes, the patient may be moved relative to the medical instrument arranged on the feed unit such that the medical instrument may travel along the trajectory in the patient or be moved or guided along the trajectory.

By moving the patient relative to the medical instrument, more space remains for moving the medical instrument along the trajectory in the patient receiving region since there is no need additionally to arrange a large intervention robot in the patient receiving region because the movement is produced by the patient table itself.

According to a further aspect of the present embodiments, the three axes are oriented such that the three axes define a three-dimensional space.

For example, the axes may each be oriented perpendicularly to one another.

For example, one axis may be oriented parallel to the direction of gravity. For example, the other two axes may then be oriented perpendicularly to the direction of gravity. In other words, two axes may be oriented horizontally and one axis vertically.

For example, one of the horizontal axes may be oriented along the patient receiving region. In other words, the axis is oriented along a longitudinal axis of the cylinder formed by the patient receiving region.

For example, the patient table may then be configured so that the patient table may be swiveled or rotated at least about the vertical axis.

The described arrangement of the axes makes it possible to provide that the patient on the patient table may be driven or moved in any possible direction. In this manner, the medical instrument may follow any possible trajectory within the patient placed on the patient table.

According to a further aspect of the present embodiments, the medical instrument is a medical needle. For example, the medical instrument is a biopsy needle.

The medical needle is configured to be inserted into the patient. For example, the medical needle may be configured to introduce a medicament and/or an active ingredient into the patient through the medical needle. Alternatively or additionally, the medical needle may be configured to take a sample (e.g., a tissue sample) from the patient. For this purpose, the medical needle may, for example, be a biopsy needle. A biopsy needle is, for example, configured for taking a biopsy.

Image-assisted interventions with a medical needle are particularly frequently carried out in a magnetic resonance tomograph. For example, taking a biopsy by the described apparatus using a biopsy needle may be carried out with image assistance particularly straightforwardly and particularly flexibly in a magnetic resonance tomograph.

According to an alternative aspect of the present embodiments, the medical instrument is a catheter or a high-intensity, focused ultrasound probe.

The catheter may, for example, be configured to insert a stent or carry out a balloon dilatation of a blood vessel or an ablation.

The high-intensity, focused ultrasound probe may, for example, be guided with a catheter. The highly focused ultrasound probe may, for example, be configured to emit high-intensity, focused ultrasound pulses. These ultrasound pulses may then, for example, be configured to carry out an ablation.

Any desired interventions in which any desired medical instrument is inserted into a patient may be carried out with the described magnetic resonance tomograph. Trajectories for various medical instruments may be followed by moving the patient table.

According to a further aspect of the present embodiments, the rail system extends along a circular arc.

The cross-section of the patient receiving region is, for example, circular. The rail system extends along a circumference of the cross-section. The rail system extends along a circular arc. The circular arc may encompass 30°, 60°, 90°, 120°, 150°, 180°, 210°, 240°, 270°, 300°, or 330° of the circumference of a circle.

The feed unit with the medical instrument may be driven along the rail system around the patient positioned on the patient table in the patient receiving region. The medical instrument may, in this manner, be driven via the feed unit along the rail system to a puncture point on the patient. The medical instrument is to be inserted into the patient at the puncture point. The medical instrument may be moved along the trajectory by moving the patient table and repositioning the medical instrument along the rail system.

According to a further aspect of the present embodiments, the rail system is of annular configuration.

The rail system forms a closed ring along the inner circumference of the inner side or patient-facing side of the patient receiving region. For example, the patient receiving region may have a circular cross-section.

The medical instrument may then be repositioned by the feed unit along the rail system around the complete circumference of the interior of the patient receiving region. It is in this manner always possible to cover the shortest distance along the rail system to a puncture point at which the medical instrument is to be inserted into the patient. It is possible in this manner to accelerate preparing for and carrying out the intervention. For example, patient comfort may be increased in this manner as the patient has the shortest possible waiting time.

According to a further aspect of the present embodiments, the magnetic resonance tomograph includes a magnet unit. The patient receiving region is enclosed by the magnet unit. The magnet unit forms an inner circumferential gap in which the rail system is arranged.

The magnet unit is configured as described above. The magnet unit is, for example, of cylindrical construction. The cylinder may have a circular or elliptical cross-section. The patient receiving region is arranged in the interior of the magnet unit. In other words, the patient receiving region is enclosed by the magnet unit.

The magnet unit is at least in part subdivided into two subportions that form an inner circumferential gap. In other words, the magnet unit is at least in part subdivided into two subportions along a cross-section of the magnet unit. The two subportions may be connected together. Alternatively, the two subportions may be independent of one another. For example, the gradient coil unit comprised by the magnet unit is subdivided into two subportions and forms the gap. For example, the radio-frequency antenna unit comprised by the magnet unit may be subdivided into two subportions and form the gap. For example, in some embodiments, the main magnet comprised by the magnet unit may be formed in one piece. In other words, the main magnet may not form a gap in some embodiments.

The gap is, for example, arranged along a circumference of the inner side of the magnet unit.

The gap is configured such that the rail system is arranged in the gap. The rail system is thus arranged between the two subportions of the magnet unit. The rail system is thus integrated in the magnet unit.

Additional space may, in this manner, be created in the patient receiving region. For example, it is possible in this manner to avoid the rail system requiring additional space in the patient receiving region. In other words, the space required in the patient receiving region by the rail system, the feed unit, and the medical instrument may be minimized in this manner.

According to a further aspect of the present embodiments, the feed unit is configured to move the medical instrument perpendicularly to the rail system.

For example, the feed unit is configured to move the medical instrument toward the patient. For example, the feed unit is configured to move the medical instrument radially inwardly into the patient receiving region. For example, the feed unit is in this manner configured to insert or introduce the medical instrument into the patient.

It is possible to achieve particularly precise movement of the medical instrument via the feed unit. This is, for example, of relevance on advancing or introducing the medical instrument. The radial movement by the feed unit may complement the movement by the patient table, and that the medical instrument may in this manner be guided very precisely along the trajectory.

According to a further aspect of the present embodiments, the feed unit is configured to rotate and/or pivot the medical instrument.

For example, the feed unit may rotate and/or pivot the medical instrument about a radial axis. The radial axis is oriented radially into the interior of the patient receiving region. For example, the radial axis is oriented perpendicularly to the rail system.

On pivoting, the medical instrument may be shifted such that the medical instrument forms an angle with the radial axis. For example, pivoting may cause the medical instrument to form an angle of between 0° and 90° with the radial axis. For example, the medical instrument may form an angle of 0° with the radial axis. The medical instrument is then oriented toward a center of the patient receiving region and, for example, perpendicularly to the rail system. The maximum angle by which the medical instrument may be pivoted by the feed unit is less than 90°.

On rotating, the medical instrument is rotated about the radial axis. For example, the pivoted medical instrument may be rotated about the radial axis.

It is possible by pivoting and/or rotating the medical instrument to provide that the medical instrument is oriented in a correct direction. In other words, it is possible by rotating and/or pivoting to provide that the medical instrument is oriented in the direction of the trajectory. In other words, it may in this manner be provided that the medical instrument is oriented in the direction of the trajectory on being inserted or introduced into the patient.

According to a further aspect of the present embodiments, the feed unit includes at least one piezo motor. The piezo motor is configured to move the medical instrument.

For example, the piezo motor is configured to move the medical instrument, as described above, perpendicularly to the rail system. The piezo motor is alternatively or additionally configured to pivot and/or rotate the medical instrument.

A piezo motor is sufficiently precise to perform or coordinate the movements of the medical instrument along the trajectory. For example, the piezo motor is sufficiently precise to guide the medical instrument during an intervention.

According to a further aspect of the present embodiments, the feed unit includes a release system. The release system is configured to detach the medical instrument from the feed unit when a force acting on the medical instrument exceeds a limit value.

The release system, for example, includes a force sensor. The force sensor is configured to detect a force acting on the medical instrument. For example, the force sensor is configured to acquire a force acting laterally on the medical instrument. In other words, the force sensor is configured to acquire a force that is not acting along the trajectory.

The release system may compare a magnitude of the measured force with the limit value. For example, the release system may compare a magnitude of a component of the force acting on the medical instrument perpendicularly to the trajectory with the limit value. The limit value is, for example, predetermined or specified.

The release system may undo or sever a connection between the feed unit and the medical instrument.

For example, once the connection has been undone or severed, the medical instrument may no longer be guided or moved with the feed unit. For example, once the connection has been undone or severed, the medical instrument is no longer firmly connected to the feed unit. Thus, once this has occurred, a relative movement of the patient table relative to the feed unit no longer results in a relative movement of the medical instrument relative to the patient table.

The release system is suitable for protecting the patient. For example, if the patient, the patient table, the rail system, and/or the feed unit moves in an unintended manner, the release system may prevent patient injury. It is, for example, possible to prevent the medical instrument from being moved away from the trajectory in the patient.

The invention further relates to a computer-implemented method for planning a movement of a medical instrument along a trajectory in a patient receiving region of an above-described magnetic resonance tomograph. The method includes a method act of receiving a trajectory. The trajectory specifies how the medical instrument is to be guided relative to a patient. The method further includes a method act of determining at least one table parameter of a table movement of patient table as a function of the trajectory. The method further includes a method act of determining at least one rail parameter of a movement of the medical instrument along the rail system as a function of the trajectory. The at least one table parameter and the at least one rail parameter are configured such that the medical instrument may be guided along the trajectory in a patient placed on the patient table by the table movement and the movement of the medical instrument along the rail system. The method also includes a method act of providing the at least one table parameter of table movement and the at least one rail parameter of the movement of the medical instrument along the rail system.

The method is, for example, configured to plan a movement of the medical instrument along the trajectory. For example, the method is configured to provide parameters (e.g., at least one table parameter and a rail parameter), with which the medical instrument may be moved or guided along the trajectory in a patient. For example, the medical instrument may be moved or guided along the trajectory by a combination of a table movement of the patient table and a movement of the medical instrument along the rail system.

The magnetic resonance tomograph with the patient receiving region is configured as described above. The medical instrument is configured as described above.

The trajectory is received by an interface. The trajectory is configured as described above. The trajectory indicates the path along which the medical instrument is to be moved or guided in a patient from a puncture site or entry point to the location of an intervention or target site.

The at least one table parameter is determined by a computing unit. The table parameter specifies a movement of the patient table in order to move or guide the medical instrument along the trajectory. The phrase “table movement” describes a movement of the patient table. The patient table is configured as described above. For example, the patient table may be displaceable along three axes and/or rotatable as described. The three axes define a three-dimensional space. The three axes are oriented as described above.

The at least one rail parameter is determined by the computing unit. Via a feed unit configured as described above, the medical instrument is connected or coupled to the rail system configured as described above or is fastened to the rail system. The position of the medical instrument may be varied by displacing or moving the feed unit along the rail system. How far or to where along the rail system the medical instrument is to be displaced along the rail system may, for example, be specified via the at least one rail parameter. For example, the puncture site may be specified by the rail parameter.

The at least one table parameter and the at least one rail parameter are determined such that the medical instrument may be guided or moved along the trajectory in a patient placed on the patient table in the patient receiving region by a movement of the patient table based on the table parameter and a movement along the rail system of the medical instrument based on the rail parameter.

The method act of providing the at least one table parameter and the at least one rail parameter provides the at least one table parameter and the at least one rail parameter using the interface. For example, the at least one table parameter and the at least one rail parameter may be provided to an above-described magnetic resonance tomograph. Alternatively, the at least one table parameter and the at least one rail parameter may be provided to a database.

Parameters (e.g., at least one table parameter and at least one rail parameter) that may bring about a movement of the medical instrument along the trajectory may be determined for a magnetic resonance tomograph configured as described above. The movement along the trajectory is possible thanks to a combination of the movement of the patient table and the movement of the medical instrument along the rail system.

According to an optional aspect of the present embodiments, a position of the patient on the patient table in the patient receiving region is taken into account when determining the at least one table parameter and when determining the at least one rail parameter.

For example, the parameters (e.g., table parameter and rail parameter) may initially be determined based on an ideal position of the patient. The parameter may then be adapted based on the real position of the patient as soon as this is known. For example, all further parameters described hereinafter for moving the medical instrument along the trajectory may be adapted.

It is initially possible when planning to start from an ideal position of the patient on the patient table. The parameter may readily be adapted to the real, actual position as soon as this is known. It is possible in this manner to take account, for example, of a slight misalignment and/or twisting of the patient on the patient table.

According to one aspect of the present embodiments, the method also includes a method act of determining at least one respiratory parameter of table movement. The respiratory parameter is configured to move the patient table in order to compensate for a respiratory movement of the patient. The method also includes a method act of providing the at least one respiratory parameter of table movement.

The at least one respiratory parameter, for example, specifies a periodic table movement synchronous with the breathing of a patient positioned on the patient table. The respiratory parameter is configured such that a tip of the medical instrument in the patient does not move relative to the patient due to the patient's breathing and so deviate from the trajectory.

The tip of the medical instrument is the part of the medical instrument that is furthest away from the connection between the feed unit and the medical instrument.

When determining the at least one respiratory parameter, it is, for example, possible to take account of sensor data from at least one sensor that acquires the patient's respiratory movement. The at least one sensor may be arranged on the patient and/or integrated in the patient table.

Alternatively, a respiratory movement of the patient may be estimated when determining the at least one respiratory parameter. For example, in the case of an intubated patient, the respiratory movement may be estimated as a function of the artificial ventilation.

The at least one respiratory parameter is provided by the interface. For example, the respiratory parameter is provided in a similar manner to the table parameter and the rail parameter.

A periodic table movement may compensate for a respiratory movement of the patient. Patient safety may, in this manner, be increased such that it is possible to avoid the medical instrument being moved to a location within the patient where the medical instrument should not be moved, so injuring the patient.

According to a further aspect of the present embodiments, the method further includes a method act of determining at least one feed parameter as a function of the trajectory. The feed unit is configured to guide the medical instrument along the trajectory using the at least one feed parameter in combination with the table movement of the patient table and the movement of the medical instrument along the rail system. The method also includes a method act of providing the at least one feed parameter.

The at least one feed parameter is determined by the computing unit. The feed unit is configured as described above to move or guide the medical instrument perpendicularly to the rail system. This movement is specified or described by the feed parameter.

The at least one feed parameter is provided by the interface in a similar manner to the at least one table parameter and the at least one rail parameter.

The entire movement may be parameterized. The medical instrument may be moved along the trajectory by a combination of movement of the patient table, movement of the medical instrument along the rail system, and a feed of the medical instrument by the feed unit.

According to an optional aspect of the present embodiments, the method includes a further method act of determining at least one rotational parameter and/or pivot parameter. The feed unit is configured to rotate the medical instrument based on the at least one rotational parameter and/or to pivot the medical instrument based on the at least one pivot parameter.

Pivoting and/or rotation is configured as described above. The at least one rotational parameter and/or the at least one pivot parameter are configured to adjust an orientation of the medical instrument by the feed unit. The orientation is configured such that the medical instrument is oriented along or tangentially to the trajectory. For example, the orientation may be corrected or adapted by the at least one rotational parameter and/or the at least one pivot parameter during movement using the at least one table parameter and the at least one rail parameter and optionally the at least one feed parameter.

The movement of the medical instrument may be planned in advance. Rotation and/or pivoting of the medical instrument by the feed unit may be necessary for this purpose.

According to a further optional aspect of the present embodiments, the method also includes a method act of controlling the magnetic resonance tomograph as a function of the at least one table parameter and the at least one rail parameter and optionally also as a function of the at least one respiratory parameter and/or the at least one feed parameter.

For example, the stated parameters are then provided to the magnetic resonance tomograph on provision. For example, the magnetic resonance tomograph may bring about or perform a movement of the patient table based on the at least one table parameter and optionally based on the at least one respiratory parameter. The magnetic resonance tomograph may also bring about a movement of the feed unit and thus of the medical instrument along the rail system based on the at least one rail parameter. The magnetic resonance tomograph may optionally bring about a movement of the medical instrument by the feed unit based on the at least one feed parameter. The medical instrument may, for example, be moved perpendicularly to the rail system by the feed unit. Alternatively or additionally, the medical instrument may be rotated and/or pivoted about an axis perpendicular to the rail system by the feed unit as a function of the at least one feed parameter.

The medical instrument may be moved along a trajectory in a patient positioned on the patient table by the described movements of the magnetic resonance tomograph. For example, the corresponding movements may also be performed without a patient being positioned on the patient table.

It is possible using the described method to determine parameters, by which the magnetic resonance tomograph may be controlled such that the medical instrument may be moved along the trajectory. For example, neither manual movement of the medical instrument nor movement by an intervention robot is necessary for this purpose. This simplifies handling and facilitates carrying out the intervention. For example, the physical prerequisites on medical personnel may be reduced in this manner. For example, the movement of the patient on the patient table reduces the space requirement for movement of the medical instrument in the patient receiving region. In this manner, an intervention by the medical instrument may also be carried out on larger patients in the magnetic resonance tomograph.

The invention also relates to a planning system for planning a movement of a medical instrument along a trajectory in a patient receiving region of an above-described magnetic resonance tomograph. The planning system includes an interface and a computing unit that are configured to perform the following method acts: receiving a trajectory that specifies how the medical instrument is to be guided relative to a patient; determining at least one table parameter of a table movement of the patient table; determining at least one rail parameter of a movement of the medical instrument along the rail system, where the at least one table parameter and the at least one rail parameter are configured such that the medical instrument is guided along the trajectory in a patient placed on the patient table by the table movement and the movement of the medical instrument; and providing the at least one table parameter of table movement and the at least one rail parameter of the movement of the medical instrument along the rail system.

Such a planning system may, for example, be configured to perform the previously described method for planning a movement of a medical instrument along a trajectory in a patient receiving region of an above-described magnetic resonance tomograph and the aspects thereof. The planning system is configured to perform this method and the aspects thereof by the interface and the computing unit being configured to perform the corresponding method acts.

The present embodiments also relate to a computer program product with a computer program and to a computer-readable medium (e.g., a non-transitory computer-readable storage medium). A largely software-based embodiment has the advantage that planning systems that are already in service may also straightforwardly be retrofitted to operate in the described manner via a software update. In addition to the computer program, such a computer program product may optionally include additional elements such as, for example, documentation and/or additional components, as well as hardware components, such as, for example, hardware keys (e.g., dongles, etc.) for using the software.

For example, the present embodiments also relate to a computer program product with a computer program that is directly loadable into a memory of planning system, with program parts for performing all the acts of the above-described method for planning a movement of a medical instrument along a trajectory in a patient receiving region of an above-described magnetic resonance tomograph and the aspects thereof when the program parts are executed by the planning system.

For example, the present embodiments relate to a computer-readable storage medium on which program parts readable and executable by a planning system are stored in order to perform all the acts of the above-described method for planning a movement of a medical instrument along a trajectory in a patient receiving region of an above-described magnetic resonance tomograph and the aspects thereof when the program parts are executed by the planning system.

The above-described properties, features, and advantages of this invention will become clearer and more readily comprehensible in connection with the following figures and the description thereof. The figures and description are not intended in any way to limit the invention and the embodiments thereof.

Same components in different figures are provided with corresponding reference signs. The figures are not in general true to scale.

### BRIEF DESCRIPTION OF THE DRAWINGS

FIG. 1 shows an example embodiment of a magnetic resonance tomograph;

FIG. 2 shows an example embodiment of a computer-implemented method for planning a movement of a medical instrument along a trajectory in a patient receiving region of a magnetic resonance tomograph; and

FIG. 3 shows an example embodiment of a planning system for planning a movement of a medical instrument along a trajectory in a patient receiving region of a magnetic resonance tomograph.

### DETAILED DESCRIPTION

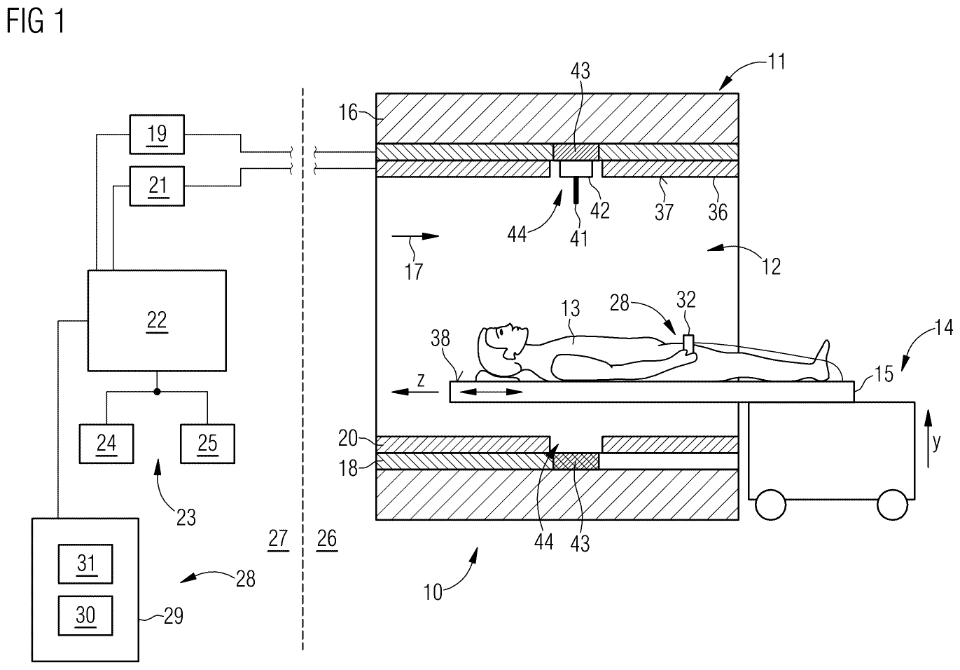

FIG. 1 shows an example embodiment of a magnetic resonance tomograph 10.

The magnetic resonance tomograph 10 includes a magnet unit 11. The magnetic resonance tomograph 10 also has a patient receiving region 12 that is configured to accommodate a patient 13. In the present example embodiment, the patient receiving region 12 is of cylindrical construction and is cylindrically surrounded in a circumferential direction by the magnet unit 11. In principle, however, a different configuration of the patient receiving region 12 may be provided. The patient 13 may be advanced and/or driven into the patient receiving region 12 by a patient positioning apparatus 14 of the magnetic resonance apparatus 10. The patient positioning apparatus 14 has a patient table 15 that is movable within the patient receiving region 12. For example, the patient table 15 is mounted movably in the direction of a longitudinal extent of the patient receiving region 12 and/or in the z direction.

In some embodiments, the patient table 15 includes at least three axes. The axes are configured such that the patient table 15 may be moved by a linear combination of a movement along and/or a rotation about the axes.

Further, the patient table 15 is, for example, mounted movably in the y direction and in the x direction. The x direction and the y direction are in each case perpendicular to the z direction. The x direction and y direction are oriented perpendicularly to one another. The x, y and z directions define the axes along which the patient table 15 is movable. The axes define a three-dimensional space.

In some embodiments, the patient table 15 is mounted such that the patient table 15 may be swiveled at least about the axis in the y direction.

The patient receiving region 12 includes an enclosure 36 that surrounds the patient receiving region 12 and has an inner side or internal wall 37. In the present example embodiment, the enclosure 36 surrounding the patient receiving region 12 is formed in one piece with the radio-frequency antenna unit 20 (e.g., with a side of the radio-frequency antenna unit 20 facing the patient receiving region 12). In an alternative configuration of the present embodiments, the enclosure 36 surrounding the patient receiving region 12 may also form a unit separate from the radio-frequency antenna unit 20.

The magnet unit 11 includes a superconductive main magnet 16 for generating a strong and, for example, constant main magnetic field 17. The magnet unit 11 also has a gradient coil unit 18 for generating magnetic field gradients that are used for spatial encoding during imaging. The gradient coil unit 18 is controlled by a gradient control unit 19 of the magnetic resonance tomograph 10. The magnet unit 11 also includes a radio-frequency antenna unit 20 for exciting polarization that is established in the main magnetic field 17 generated by the main magnet 16. The radio-frequency antenna unit 20 is controlled by a radio-frequency antenna control unit 21 of the magnetic resonance tomograph 10 and emits radio-frequency magnetic resonance sequences into the patient receiving region 12 of the magnetic resonance tomograph 10.

The magnetic resonance tomograph 10 has a system control unit 22 for controlling the main magnet 16 and the gradient control unit 19 and for controlling the radio-frequency antenna control unit 21. The system control unit 22 is comprised by a computing unit of the magnetic resonance tomograph 10. The system control unit 22 provides central control of the magnetic resonance tomograph 10, such as, for example, carrying out a predetermined imaging gradient echo sequence. In addition, the system control unit 22 includes an evaluation unit, not shown greater detail, for evaluating medical image data or magnetic resonance tomograph (MRI) image data that is acquired during the examination or magnetic resonance examination.

The magnetic resonance tomograph 10 also includes a user interface 23 that is connected to the system control unit 22. Control information, such as, for example, imaging parameters, and reconstructed MRI image data may be displayed on a display unit 24 (e.g., on at least one monitor) of the user interface 23 for a medical operator or medical personnel. The user interface 23 also has an input unit 25, by which information and/or parameters may be input by the medical operator during a measurement procedure.

The magnet unit 11 of the magnetic resonance apparatus 10 is arranged together with the patient positioning apparatus 14 within an MRI room 26. The system control unit 22 is arranged together with the user interface 23 within a control room 27. The control room 27 is separate from the MRI room 26. For example, the MRI room 26 is shielded from the control room 27 with regard to radio-frequency radiation. During an examination, the patient 13 is located within the MRI room 26, while the medical operator is mainly located within the control room 27. The MRI room 26 and control room 27 together form the MRI examination room.

The magnetic resonance tomograph 10 has a communication unit 28 for communication and/or exchange of information between the patient 13 and the medical operator during an examination. On the operator side, the communication unit 28 has a communication element configured as an operator console 29. The communication element (e.g., the operator console 29) may be arranged within the control room 27. The operator console 29 has an input element 30 and an output element 31. The input element 30 and/or the output element 31 may be configured as an acoustic and/or visual input element 30 and/or output element 31.

On the patient side, the communication unit 28 also has a first communication element configured as an input element 32. Using the input element 32, the patient 13 may inform the operator (e.g., the medical operator) of a sensation such as, for example, discomfort during the examination. In the present example embodiment, the input element 32 is configured as a patient squeeze ball or alarm ball. However, further input elements 32 as may appear appropriate to a person skilled in the art, such as, for example, a microphone, etc., are possible in a further embodiment of the communication unit 28.

The magnetic resonance tomograph 10 also includes a rail system 43 configured to guide a feed unit 42. The feed unit 42 is configured to hold a medical instrument 41. The feed unit 42 is connected to the medical instrument 41. In other words, the medical instrument 41 is fastened to the feed unit 42. The feed unit 42 may be repositioned along the rail system 43. The rail system 43, for example, extends along a circular arc on the patient-facing inner side of the enclosure 36. The circular arc may, for example, encompass an angle of 10°, 20°, 30°, 40°, 50°, 60°, 70°, 80°, 90°, 120°, 180°, 210°, 240°, or 270°. For example, the rail system 43 may be of annular configuration (e.g., encompass a circular arc of) 360°.

The feed unit 42 may, for example, be movable along the rail system 43 by rollers. Alternatively, the feed unit 42 may be movable along the rail system 43 using other bearings.

In the example embodiment shown, the rail system 43 is arranged in an inner circumferential gap 44 of the magnet unit 11. For example, the rail system 43 is arranged in an inner circumferential gap 44 that is formed by the gradient coil unit 18 and the radio-frequency antenna unit 20. The gradient coil unit 18 and the radio-frequency antenna unit 20 are subdivided in two subportions that are spaced apart from one another by the inner circumferential gap 44. Alternatively, the rail system 43 may be arranged along the internal wall 37 of the enclosure 36 of the patient receiving region 12.

The medical instrument 41 is configured to carry out an intervention. The medical instrument 41 is, for example, a medical needle (e.g., a biopsy needle). In this case, the intervention is, for example, a biopsy. Alternatively, the medical instrument 41 may be a catheter and/or a high-intensity, focused ultrasound probe.

In some embodiments, the feed unit 42 is configured to shift the medical instrument 41 radially, perpendicularly to the rail system 43. In other words, the feed unit 42 may move the medical instrument 41 toward the patient 13. For example, the feed unit 42 may move the medical instrument 41 with a piezo motor.

In some embodiments, the feed unit 42 is configured to rotate the medical instrument 41 about an axis perpendicular to the rail system 43 and/or to pivot the medical instrument 41 relative to this axis. In this manner, the orientation of the medical instrument 41 relative to the patient 13 may be adapted. For example, the feed unit 42 may rotate and/or pivot the medical instrument 41 using a piezo motor.

The feed unit 42 may, for example, include a release system. The release system includes a force sensor that measures or acquires a force acting on the medical instrument 41. For example, the force sensor measures a force acting on the medical instrument 41 perpendicularly to the trajectory. The release system detaches the medical instrument 41 from the feed unit 42 when this force exceeds a limit value.

The magnetic resonance tomograph 10 is configured to guide or move the medical instrument 41 along a specified trajectory in the patient 13 by a movement of the patient table 15 along the above-described axes and/or a rotation about at least one of the above-described axes and a movement of the medical instrument 41 along the rail system 43 by the feed unit 42.

The magnetic resonance tomograph 10 shown may include further components that magnetic resonance tomographs 10 usually have. A general mode of operation of an MRI system 1 is additionally known to a person skilled in the art, and therefore, no detailed description of the further components is provided.

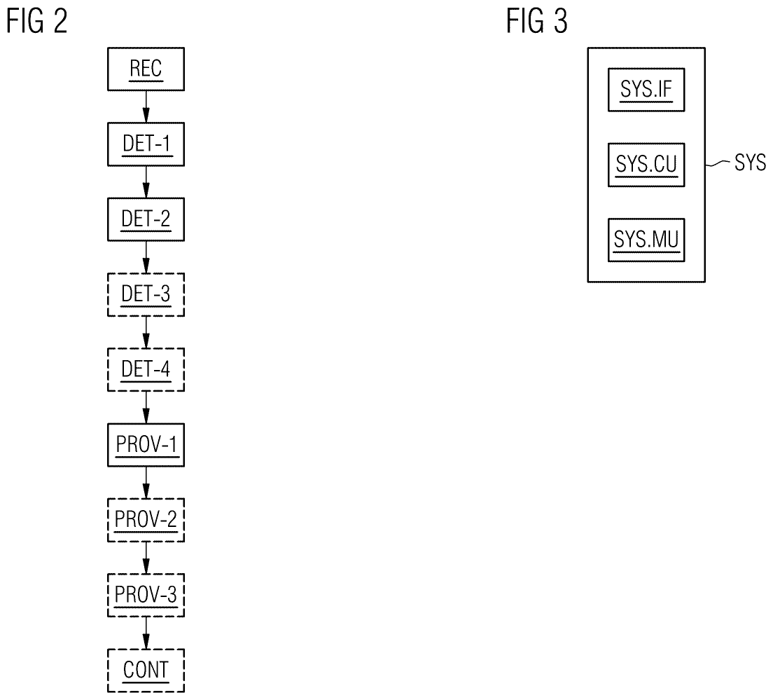

FIG. 2 shows an example embodiment of a computer-implemented method for planning a movement of a medical instrument 41 along a trajectory in a patient receiving region 12 of a magnetic resonance tomograph 10.

The magnetic resonance tomograph 10 is configured as described in relation to FIG. 1.

In a method act of receiving REC the trajectory, a trajectory is received by an interface SYS.IF. The trajectory specifies how the medical instruments 41 is to be guided relative to a patient 13. In other words, the trajectory specifies a path for the medical instrument 41 within the patient 13. In other words, the trajectory specifies a path of the medical instrument 41 from a puncture site into the patient 13 up to a target site. The path may be straight or curved. Alternatively, the path may extend along a blood vessel. The target site is the location in the patient 13 where an intervention is to be carried out with the assistance of the medical instrument 41. The medical instrument 41 is configured as described in relation to FIG. 1.

In a method act of determining DET-1 at least one table parameter of a table movement of the patient table 15 of the magnetic resonance tomograph 10, the at least one table parameter is determined by a computing unit SYS.CU. The patient table 15 is configured as described in relation to FIG. 1. For example, the patient table 15 is configured to be movable along at least three axes. For example, the patient table 15 may be configured to be rotatable about at least one of the axes. The at least one table parameter describes a movement of the patient table 15 along these axes and/or a rotation of the patient table 15 about these axes. The at least one table parameter is determined based on the trajectory.

In a method act of determining DET-2 at least one rail parameter of a movement of the medical instrument 41 along the rail system 43, the at least one rail parameter is determined by the computing unit SYS.CU. The rail system 43 is configured as described in relation to FIG. 1. The medical instrument 41 is fastened to the feed unit 42 as described in relation to FIG. 1. The feed unit 42 may be shifted or moved or guided along the rail system 43. As a result of the feed unit 42 being moved or shifted or guided, the medical instrument 41 is also shifted or moved or guided along the rail system 43. The at least one rail parameter describes the movement of the medical instrument 41 along the rail system 43. For example, the at least one rail parameter may specify at least one position of the medical instrument 41 along the rail system. The at least one rail parameter is determined based on the trajectory.

The at least one table parameter and the at least one rail parameter are configured such that the medical instrument 41 may be moved or guided along the trajectory in the patient 13 placed on the patient table 15 via the table movement resulting from the parameter and the movement of the medical instrument 41 along the rail system 43.

In a further method act of providing PROV-1 the at least one table parameter and the at least one rail parameter, the two parameters are provided by an interface SYS.IF. For example, the two parameters are provided to the magnetic resonance tomograph 10. For example, the two parameters may be provided such that the magnetic resonance tomograph 10 may perform the table movement and the movement of the medical instrument 41 along the rail system 43 based on the at least one table parameter and the at least one rail parameter.

The at least one table parameter and the at least one rail parameter are, for example, determined and provided in advance of an intervention.

In an optional method act of determining DET-3 at least one respiratory parameter of table movement, at least one respiratory parameter is determined by the computing unit SYS.CU. The at least one respiratory parameter specifies a table movement that is overlaid on the table movement based on the at least one table parameter. The table movement based on the at least one respiratory parameter is configured such that a respiratory movement of the patient 13 is compensated for by the table movement based on the at least one respiratory parameter. In other words, the table movement based on the at least one respiratory parameter is complementary to the movement of the patient 13 that is caused by breathing or respiratory movement of the patient 13.

The at least one respiratory parameter may, for example, be determined while the intervention is being carried out. For example, the at least one respiratory parameter may be determined based on sensor data. The sensor data may be acquired by at least one sensor that is fastened to the patient 13. Alternatively, the at least one respiratory parameter may be determined in advance of the intervention. For example, the at least one respiratory parameter may be determined based on estimates of respiratory movement of the patient. For example, the at least one respiratory parameter may be based on a periodic estimate. Alternatively, the at least one respiratory parameter may be determined using a trained function.

In a further optional method act of providing PROV-2, the at least one respiratory parameter is provided via the interface SYS.IF. For example, the at least one respiratory parameter is provided to the magnetic resonance tomograph 10 such that the patient table 15 may be moved based on the at least one respiratory parameter. Alternatively, the at least one respiratory parameter may be provided to a database. For example, the magnetic resonance tomograph 10 may then retrieve the respiratory parameter from the database.

In a further optional method act of determining DET-4 at least one feed parameter, the at least one feed parameter is determined by the computing unit SYS.CU. The at least one feed parameter is determined based on the trajectory. The feed unit 42 is configured to guide the medical instrument 41 along the trajectory using the at least one feed parameter in combination with the table movement of the patient table 15 based on the at least one table parameter and the movement of the medical instrument 41 along the rail system 43 based on the at least one rail parameter.

For example, the at least one feed parameter may define a movement of the medical instrument 41 perpendicular to the rail system 43. The movement perpendicular to the rail system 43 may, for example, be performed by a piezo motor. For example, the feed unit 42 then includes the piezo motor.

In some embodiments, the at least one feed parameter may alternatively or additionally define a rotation and/or pivoting of the medical instrument 41 about or relative to an axis perpendicular to the rail system 43 via the feed unit 42. For example, the feed unit 42 may include a piezo motor to perform the rotation and/or pivoting.

The at least one feed parameter may be determined in advance of the intervention.

In a further optional method act of providing PROV-3 the at least one feed parameter, the at least one feed parameter is provided by the interface SYS.IF. For example, the at least one feed parameter is provided such that the medical instrument 41 may be moved along the trajectory in the patient 13 based on the at least one feed parameter, the at least one table parameter, and the at least one rail parameter.

In a further optional method act of controlling CONT the magnetic resonance tomograph 10 based on the at least one table parameter, the at least one rail parameter, and optionally additionally based on the at least one feed parameter and/or the at least one respiratory parameter, the magnetic resonance tomograph 10 is controlled based on the provided parameters. For example, the movement of the patient table 15 or the table movement and the movement of the medical instrument 41 along the rail system 43 and optionally a movement of the medical instrument 41 are controlled by the feed unit 42. The movements are controlled such that the medical instrument 41 is moved along the trajectory in the patient 13 when a patient 13 is positioned on the patient table 15.

FIG. 3 shows a planning system for planning a movement of a medical instrument 41 along a trajectory in a patient receiving region 12 of a magnetic resonance tomograph 10.

The planning system SYS shown for planning a movement of a medical instrument 41 along a trajectory in a patient receiving region 12 of a magnetic resonance tomograph 10 is configured to perform a method according to the present embodiments for planning a movement of a medical instrument 41 in a patient receiving region 12 of a magnetic resonance tomograph 10. The planning system SYS includes an interface SYS.IF, a computing unit SYS.CU, and a memory unit SYS.MU.

The planning system SYS may, for example, be a computer, a microcontroller, or an integrated circuit (IC). Alternatively, the planning system SYS may be a real or virtual computer network (e.g., a technical name for a real computer network is “cluster,” and a technical name for a virtual computer network is “cloud”). The planning system SYS may be configured as a virtual system that is executed on a computer or a real computer network or a virtual computer network (e.g., a technical name is “virtualization”).

The interface SYS.IF may be a hardware or software interface (e.g., a PCI bus, USB or FireWire). The computing unit SYS.CU may include hardware and/or software components (e.g., a microprocessor or a field programmable gate array (FPGA)). The memory unit SYS.MU may be configured as a volatile working memory (e.g., random access memory (RAM)) or as a non-volatile mass storage device (e.g., hard disk, USB stick, SD card, solid state disk (SSD)).

The interface SYS.IF may, for example, include a plurality of subinterfaces that perform different method acts of the respective method according to the present embodiments. In other words, the interface SYS.IF may be configured as a plurality of interfaces SYS.IF. The computing unit SYS.CU may, for example, include a plurality of subcomputing units that perform different method acts of the respective method according to the present embodiments. In other words, the computing unit SYS.CU may be configured as a plurality of computing units SYS.CU.

Where it has not yet been explicitly done but is reasonable and in line with the purposes of the invention, individual example embodiments, individual subaspects, or features thereof may be combined with one another or interchanged without going beyond the scope of the present invention. Advantages of the invention described in relation to one example embodiment also apply, where transferable, to other example embodiments without being explicitly stated to do so.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A magnetic resonance tomograph comprising:
a patient table;
a patient receiving region;
a feed unit that is configured to guide a medical instrument; and
a rail system for guiding the feed unit, the rail system running extensively on an interior side along an inner side of the patient receiving region,
wherein the patient table is configured to position a patient in the patient receiving region, and
wherein the patient table is further configured to be movable such that the medical instrument is guideable along a trajectory in the patient with the assistance of a movement of the patient table.

2. The magnetic resonance tomograph of claim 1, wherein the patient table comprises at least three axes,
wherein the at least three axes are configured such that the medical instrument is movable along the trajectory by a movement of the patient table, and
wherein the movement of the patient table is produced by a linear combination of movements along the at least three axes, a rotation about at least one of the at least three axes, or the linear combination of movements and the rotation.

3. The magnetic resonance tomograph of claim 1, wherein the at least three axes are oriented such that the at least three axes define a three-dimensional space.

4. The magnetic resonance tomograph of claim 1, wherein the medical instrument is a medical needle.

5. The magnetic resonance tomograph of claim 4, wherein the medical needle is a biopsy needle.

6. The magnetic resonance tomograph of claim 1, wherein the rail system extends along a circular arc.

7. The magnetic resonance tomograph of claim 1, wherein the rail system is of annular configuration.

8. The magnetic resonance tomograph of claim 1, wherein the magnetic resonance tomograph further comprises:
a magnet unit,
wherein the patient receiving region is enclosed by the magnet unit,
wherein the magnet unit forms an inner circumferential gap, and
wherein the rail system is arranged in the inner circumferential gap.

9. The magnetic resonance tomograph of claim 1, wherein the feed unit is configured to move the medical instrument perpendicularly to the rail system.

10. The magnetic resonance tomograph of claim 1, wherein the feed unit is configured to rotate, pivot, or rotate and pivot the medical instrument.

11. The magnetic resonance tomograph of claim 8, wherein the feed unit comprises at least one piezo motor that is configured to move the medical instrument.

12. The magnetic resonance tomograph of claim 1, wherein the feed unit comprises a release system, and
wherein the release system is configured to detach the medical instrument from the feed unit when a force acting on the medical instrument exceeds a limit value.

13. A method for planning a movement of a medical instrument along a trajectory in a patient receiving region of a magnetic resonance tomograph, the method being computer-implemented and comprising:
receiving a trajectory, wherein the trajectory specifies how the medical instrument is to be guided relative to a patient;
determining at least one table parameter of a table movement of a patient table as a function of the trajectory;
determining at least one rail parameter of a movement of the medical instrument along a rail system of the magnetic resonance tomograph as a function of the trajectory, wherein the at least one table parameter and the at least one rail parameter are configured such that the medical instrument is guideable along the trajectory in a patient placed on the patient table by the table movement and the movement of the medical instrument along the rail system; and
providing the at least one table parameter of the table movement and the at least one rail parameter of the movement of the medical instrument along the rail system.

14. The method of claim 13, further comprising:
determining at least one respiratory parameter of table movement, wherein the at least one respiratory parameter is configured to move the patient table in order to compensate for a respiratory movement of the patient; and
providing the at least one respiratory parameter of the table movement.

15. The method of claim 13, further comprising:
determining at least one feed parameter as a function of the trajectory, wherein a feed unit of the magnetic resonance tomograph is configured to guide the medical instrument along the trajectory using the at least one feed parameter in combination with the table movement of the patient table and the movement of the medical instrument along the rail system; and
providing the at least one feed parameter.

16. A planning system for planning a movement of a medical instrument along a trajectory in a patient receiving region of a magnetic resonance tomograph, the planning system comprising:
an interface; and
a computing unit,
wherein the interface and the computing unit are configured to:
receive a trajectory, wherein the trajectory specifies how the medical instrument is to be guided relative to a patient;
determine at least one table parameter of a table movement of a patient table of the magnetic resonance tomograph; and
determine at least one rail parameter of a movement of the medical instrument along a rail system of the magnetic resonance tomograph, wherein the at least one table parameter and the at least one rail parameter are configured such that the medical instrument is guideable along the trajectory in a patient placed on the patient table by the table movement and the movement of the medical instrument along the rail system; and
provide the at least one table parameter of table movement and the at least one rail parameter of the movement of the medical instrument along the rail system.

17. In a non-transitory computer-readable storage medium that stores instructions executable by a planning system to plan a movement of a medical instrument along a trajectory in a patient receiving region of a magnetic resonance tomograph, the instructions comprising:
receiving a trajectory, wherein the trajectory specifies how the medical instrument is to be guided relative to a patient;
determining at least one table parameter of a table movement of a patient table as a function of the trajectory;
determining at least one rail parameter of a movement of the medical instrument along the rail system as a function of the trajectory, wherein the at least one table parameter and the at least one rail parameter are configured such that the medical instrument is guideable along the trajectory in a patient placed on the patient table by the table movement and the movement of the medical instrument along the rail system; and
providing the at least one table parameter of the table movement and the at least one rail parameter of the movement of the medical instrument along the rail system.
